(19) United States
(12) Patent Application Publication
(10) Pub. No.: US 2015/0147769 A1
(43) Pub. Date: May 28, 2015

(54) METHOD OF TESTING SAMPLE AND MICROFLUIDIC DEVICE

(71) Applicant: SAMSUNG ELECTRONICS CO., LTD., Suwon-si (KR)

(72) Inventors: Kui Hyun KIM, Hwaseong-si (KR); Taguchi TAKAYUKI, Tokushima-shi (JP); Sung Joon PARK, Suwon-si (KR); Chang Soo PARK, Hwaseong-si (KR); Sang Hyun BAEK, Hwaseong-si (KR); Tae Han LEE, Seoul (KR)

(73) Assignee: SAMSUNG ELECTRONICS CO., LTD., Suwon-si (KR)

(21) Appl. No.: 14/528,157

(22) Filed: Oct. 30, 2014

(30) Foreign Application Priority Data
Nov. 22, 2013 (KR) 10-2013-0142975

(51) Int. Cl. C12Q 1/40; G01N 21/78

(52) U.S. Cl. CPC C12Q 1/40; G01N 21/78; G01N 2201/02
USPC 435/22; 436/124; 422/419; 435/288.7

(57) ABSTRACT

A method of testing a sample to determine a concentration of a target material included in the sample and a microfluidic device in which a reaction of the sample and a reagent occurs are provided. The method includes mixing a sample with a reagent that changes optical characteristics in accordance with a concentration of chlorine ions in the sample, and a capturing material that captures some of the chlorine ions in the sample; measuring the optical characteristics after mixing the sample with the reagent and the capturing material; and determining a concentration of the chlorine ions in the sample based on the measured optical characteristics.

### CROSS-REFERENCE TO RELATED APPLICATION

This application claims priority from Korean Patent Application No. 10-2013-0142975, filed on Nov. 22, 2013 in the Korean Intellectual Property Office, the disclosure of which is incorporated herein by reference.

### BACKGROUND

1. Field

Apparatuses and methods consistent with exemplary embodiments relate to a method of testing a sample to determine a concentration of a target material included in the sample and a microfluidic device in which a reaction of the sample and a reagent occurs.

2. Description of the Related Art

Recently, compact and automated equipment capable of instantly analyzing a sample has been developed in various fields including environment monitoring, food inspection, medical diagnosis, etc.

Particularly, to measure the concentration of a target material included in a sample for medical diagnosis, an enzyme activated by the target material and/or a substrate degraded by the enzyme may be included in a reagent. Optical characteristics shown by the degradation of the substrate may be measured, thereby estimating the amount of the activated enzyme, and thus, the concentration of the target material.

However, the optical characteristics cannot be discriminated in a concentration range corresponding to a dynamic range of the target material, so development of a method of enhancing concentration discrimination in the dynamic range is necessary.

### SUMMARY

One or more exemplary embodiments provide a method of testing a sample capable of enhancing concentration discrimination in a high concentration range of a target material without employing a separate step or structure for diluting the sample, and a microfluidic device used therefor.

In accordance with an aspect of an exemplary embodiment, there is provided a method of determining a concentration of chlorine ions in a sample, the method including: mixing a sample, a reagent that changes optical characteristics change in accordance with a concentration of chlorine ions in the sample, and a capturing material that captures some of the chlorine ions in the sample, measuring the optical characteristics after mixing the sample with the reagent and the capturing material, and determining the concentration of the chlorine ions in the sample based on the measured optical characteristics.

The capturing material may be a compound including an amine (—NH₂) group.

The capturing material may be at least one selected from the group consisting of urea, thio-urea, an N-(2-acetamido)-2-aminoethanesulfonic acid (ACES) buffer and a 2-[(2-amino-2-oxoethyl)-(carboxymethyl)amino]acetic acid (ADA) buffer.

The amine group of the capturing material may bind to the chlorine ions.

The reagent may include an enzyme activated by the chlorine ions and a substrate degraded by the activated enzyme.

The enzyme may be activated by chlorine ions that are not bound by the capturing material.

The enzyme may be α-amylase.

The substrate may be 2-chloro-4-nitrophenyl-alpha-maltotrioside (CNPG3).

The CNPG3 may be hydrolyzed by the α-amylase to generate 2-chloro-4-nitrophenol (CNP) and α-maltotriose (G3).

In accordance with an aspect of another exemplary embodiment, there is provided a microfluidic device including at least one chamber containing a reagent that changes optical characteristics according to a concentration of chlorine ions in a sample, and a capturing material that captures some of the chlorine ions in the sample, and a sample inlet into which the sample is injected.

The capturing material may be a compound including an amine (—NH₂) group.

The capturing material may be at least one selected from the group consisting of urea, thio-urea, an ACES buffer, and an ADA buffer.

The amine group of the capturing material may bind to the chlorine ions.

The reagent may include an enzyme activated by the chlorine ions and a substrate degraded by the activated enzyme.

The enzyme may be activated by chlorine ions that are not bound to the capturing material.

The enzyme, the substrate and the capturing material may be contained in one of the at least one chambers.

A channel connecting the at least one chamber with the sample inlet may be further included.

The enzyme may be α-amylase.

The substrate may be CNPG3.

### BRIEF DESCRIPTION OF THE DRAWINGS

The above and/or other aspects will become apparent and more readily appreciated from the following description of exemplary embodiments, taken in conjunction with the accompanying drawings of which:

FIG. 1 is a graph showing optical intensity values per concentration of chlorine ions;

FIG. 2 is a flowchart showing a method of testing a sample in accordance with an exemplary embodiment;

FIG. 3 is a schematic diagram showing a reaction occurring when a sample and a reagent are mixed according to a method of testing a sample in accordance with an exemplary embodiment;

FIG. 4 is a schematic diagram showing a reaction occurring when a reagent including urea and a sample are mixed according to the method of testing a sample in accordance with an exemplary embodiment;

FIG. 5 is a flowchart schematically showing the steps involved in a reaction occurring when a reagent including urea and a sample are mixed according to the method of testing a sample in accordance with an exemplary embodiment;

FIG. 6 is an absorbance graph measured by adding thio-urea to a capturing material according to the method of testing a sample in accordance with an exemplary embodiment;

FIG. 7 is a graph showing a comparison of test results between performing the method of testing including adding thio-urea and not adding thio-urea to a capturing material;

FIG. 8 is an exterior view of a microfluidic device in accordance with an exemplary embodiment;

FIG. 9 is an exploded perspective view of a structure of a testing unit of the microfluidic device shown in FIG. 8;

FIG. 10 is an exterior view of a testing device capable of measuring test results using the microfluidic device in accordance with an exemplary embodiment;

FIG. 11 is a top view of a microfluidic device in accordance with another exemplary embodiment; and

FIG. 12 is an exterior view of a testing device for measuring test results using the microfluidic device in accordance with another exemplary embodiment.

### DETAILED DESCRIPTION

Exemplary embodiments will now be described in detail with reference to the accompanying drawings, wherein like reference numerals refer to like elements throughout.

Among various methods of determining a concentration of a target material included in a sample, there is a method involving use of an enzyme activated by a target material and a substrate degraded by the activated enzyme. As a specific example, an enzyme method used in an electrolyte test may be used. The method may include use of α-amylase and 2-chloro-4-nitrophenyl-α-D-maltotrioside (CNPG3) as an enzyme and a substrate, respectively, to determine a concentration of electrolyte ions, such as chlorine (Cl⁻) ions.

A reaction mechanism for determining the concentration of chlorine ions using α-amylase and CNPG3 is as follows.

α-Amylase+Cl⁻

CNPG3→CNP+G3

Referring to the reaction mechanism, the chlorine ions (Cl⁻) activate the α-amylase, and the activated α-amylase hydrolyzes the CNPG3, thereby generating 2-chloro-p-nitrophenol (CNP) and α-maltotriose (G3).

CNP is a coloring material, which provides the ability to estimate the amount of activated α-amylase by measuring the optical characteristics shown by the CNP. Additionally, the concentration of chlorine ions may be determined from the amount of the activated α-amylase. As such, the concentration of the chlorine ions may be determined from the optical characteristics caused by the CNP.

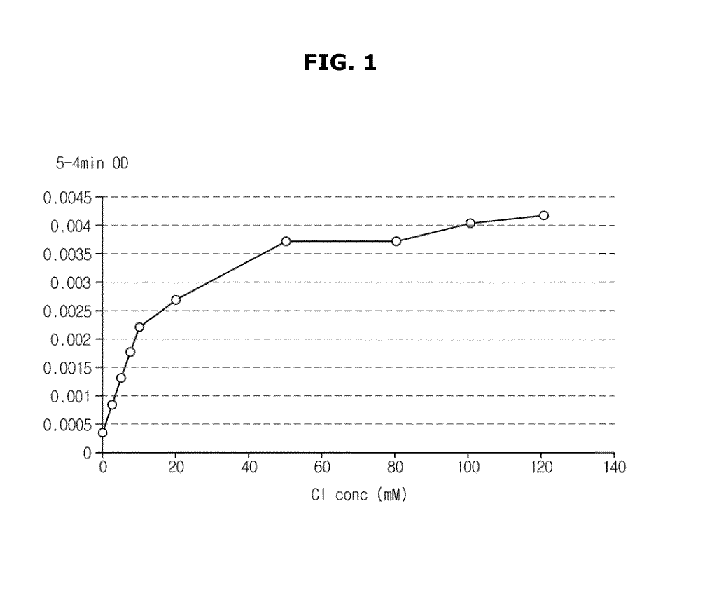

FIG. 1 is a graph showing optical intensity values per concentration of chlorine ions. The graph of FIG. 1 is a result obtained by adding α-amylase and CNPG3 to a sample including chlorine ions.

Referring to FIG. 1, it can be seen that while the slope of the optical density value is increased in the lower concentration range of the chlorine ions, and discrimination between concentrations is high. However, the slope of the optical density value is close to 0 in the higher concentration range of the chlorine ions, resulting in the discrimination between concentrations being very low.

As shown in FIG. 1, when the concentration of chlorine ions present in a biological sample is measured, a dynamic range is from 80 to 135 mM. Since the discrimination between concentrations in the dynamic range is very high, the optical density value was measured by diluting the sample to reduce the concentration of chlorine ions in the sample, and thereafter, adding α-amylase and CNPG3. Thus, to use the diluted sample in the test, a step for diluting the sample must be added, and a separate systemic structure for diluting the sample is needed.

However, the method of testing a sample in accordance with an exemplary embodiment provided herein provides enhanced discrimination between concentrations of the target material without adding a separate step or a systemic structure for diluting the sample or using a capturing material for capturing a target material.

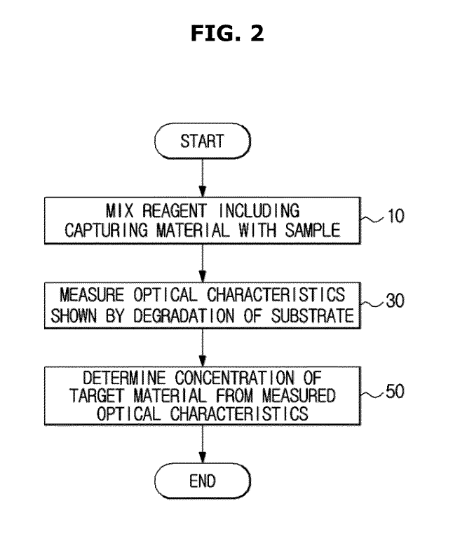

FIG. 2 is a flowchart showing a method of testing a sample in accordance with an exemplary embodiment.

Referring to FIG. 2, first, a reagent including a capturing material and a sample are mixed (10). The reagent may be used to induce a change in optical characteristics according to the concentration of a target material present in the sample. As an example, the reagent may include an enzyme activated by the target material in the sample and a substrate degraded by the enzyme to change optical characteristics thereof. In various embodiments, the type of capturing material used may depend on the type of target material present in the sample. Descriptions of specific materials useful in the reagent will be provided below.

When the mixed reagent and sample react, optical characteristics of a reaction product change according to the concentration of the target material. Thus, the optical characteristics shown by degradation of the substrate are measured (30). Exemplary optical characteristics suitable for measuring in the test method include, but are not limited to, absorbance, transmittance, reflectivity, and luminescence. Thus, suitable optical characteristics may be measured according to the type of test being performed and/or the type of device used to perform the test.

Thereafter, the concentration of the target material is determined from the measured optical characteristics (50). When the sample includes an enzyme and a substrate according to the above-described example, the change in optical characteristics may be the result of degrading the substrate by an activated enzyme, which may be activated by the target material. Accordingly, the concentration of the target material may be determined by analyzing the measured optical characteristics.

Since some of the target material present in the sample binds to the capturing material and thus does not participate in activation of an enzyme, an effect similar to dilution of the target material may be obtained. That is, the effect of enhancement in discrimination between concentrations may also be obtained in a high concentration range.

Hereinafter, a composition of a reagent mixed with the target material and a mechanism of binding the capturing material included in the reagent to the target material will be explained in detail.

The method of testing a sample in accordance with an exemplary embodiment may be applied in various fields including medical diagnosis, environment inspection, etc. Particularly, in medical diagnosis, when an electrolyte test is performed, the concentration of chlorine ions, for example, may be determined through the above-described method of testing. Thus, for explanatory purposes only, the exemplary embodiment will be described below using chlorine ions as a target material.

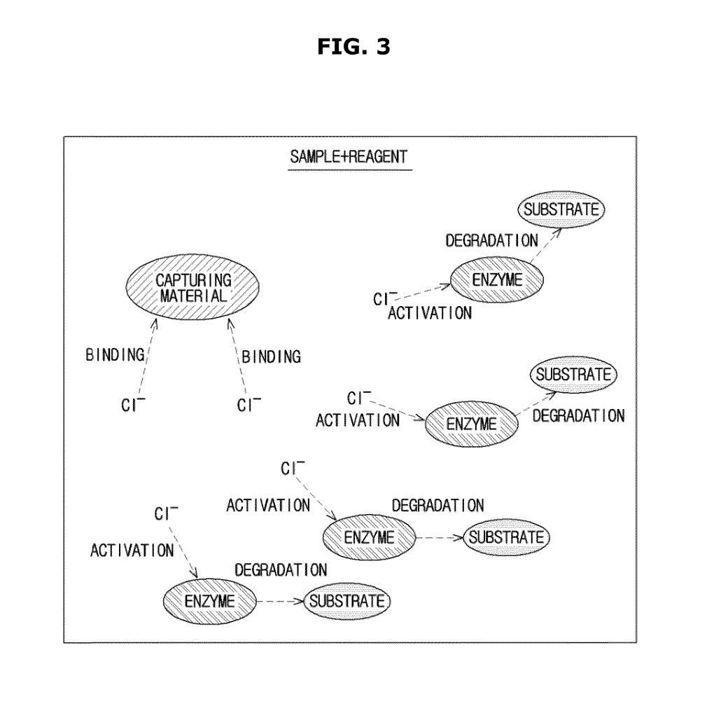

FIG. 3 is a schematic diagram showing a reaction occurring when a sample and a reagent are mixed according to the method of testing a sample in accordance with an exemplary embodiment.

As described above, an enzyme and a substrate may be used to measure the concentration of chlorine ions. When a reagent including a capturing material, an enzyme, and a substrate is added to a sample containing chlorine ions, as shown in FIG. 3, the capturing material binds to some of the chlorine ions contained in the sample, while the chlorine ions to which the capturing material does not bind activate the enzyme.

The activated enzyme then degrades the substrate, thereby changing optical characteristics. Since some of the chlorine ions within the sample do not participate in the activation of the enzyme as a result of binding to the capturing material, a similar effect to dilution may be obtained, thereby enhancing discrimination between concentrations in the dynamic range of the chlorine ions.

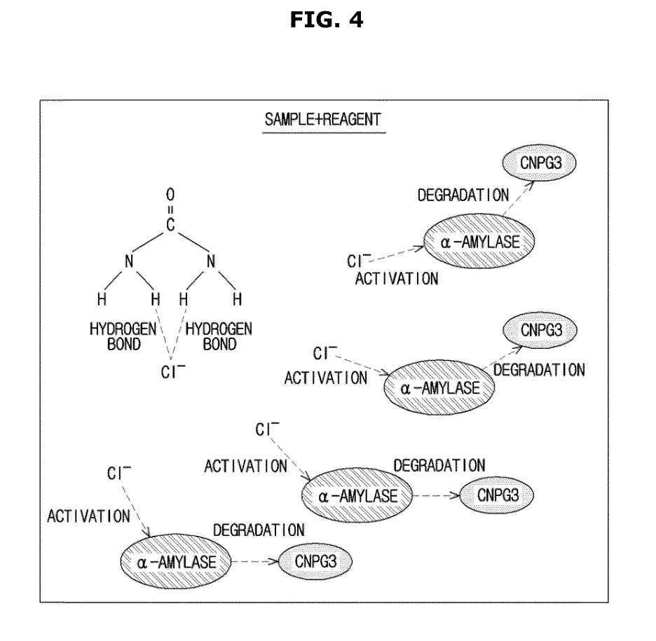

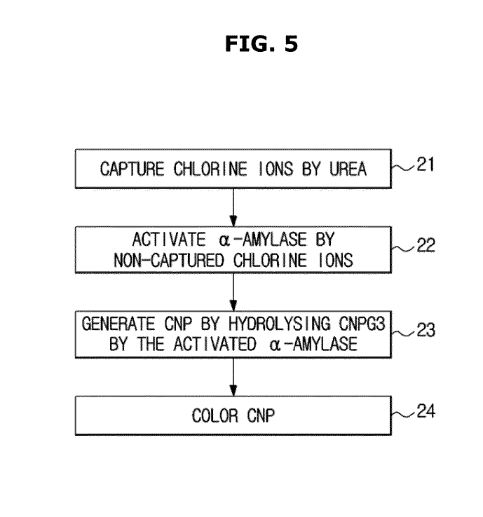

FIG. 4 is a schematic diagram showing a reaction occurring when a sample including urea and a reagent are mixed according to the method of testing a sample in accordance with an exemplary embodiment, and FIG. 5 is a flowchart schematically showing the steps involved in the reaction.

Exemplary capturing materials capable of binding to chlorine ions include, but are not limited to, compounds having an amine group, such as, for example, urea or thio-urea. Urea has the formula: CO(NH₂)₂, and thio-urea has the formula: CS(NH₂)₂, which is formed by substituting an oxygen atom of urea with a sulfur atom. In FIGS. 4 and 5, urea is used as the capturing material, the enzyme is α-amylase, and CNPG3 is used as the substrate.

Referring to FIGS. 4 and 5, when the sample and the reagent are mixed, the urea captures a predetermined amount of chlorine ions present in the sample (21). The capturing of the chlorine ions occurs when an amine (—NH₂) group of the urea binds to the chlorine ions. As such, the amount of bound chlorine ions changes according to the amount of urea included in the reagent.

Particularly, because an electron of a hydrogen (H) atom is attracted to a negatively charged nitrogen (N) atom in the amine group of the urea, the hydrogen atom becomes positive. Thereafter, a negatively charged chlorine ion approaches the electrically positive hydrogen atom, forming a hydrogen bond as shown in FIG. 4. That is, the chlorine ions are captured due to the hydrogen bond.

The chlorine ions to which the urea binds therefore do not participate in activation of α-amylase, and only non-captured chlorine ions activate the α-amylase (22).

The activated α-amylase hydrolyzes CNPG3, thereby generating CNP (23). Thus, a reaction mechanism for generating CNP and G3 by hydrolyzing CNPG3 is described above.

Since the CNP is colored (24), as described in the flowchart of FIG. 2, the optical characteristics exhibited by degrading the substrate may be measured (S30), thereby determining the concentration of chlorine ions as a target material (S50).

When a predetermined amount of the urea is mixed with the sample, the urea binds to a predetermined amount of chlorine ions present in the sample. Accordingly, when a binding ratio between the urea and the chlorine ions is found (i.e., the amount of chlorine ions binding to one urea molecule and the total amount of urea), the amount of the chlorine ions not participating in the activation of the α-amylase due to being bound by the urea may be determined. Consequently, the concentration of the chlorine ions present in the sample may be determined.

Thio-urea may also be used to simulate the effect of diluting the sample by capturing chlorine ions in the same manner as described above.

Additional examples of capturing materials that bind to chlorine ions, are an ACES buffer represented by Structural Formula 1, and an ADA buffer represented by Structural Formula 2.

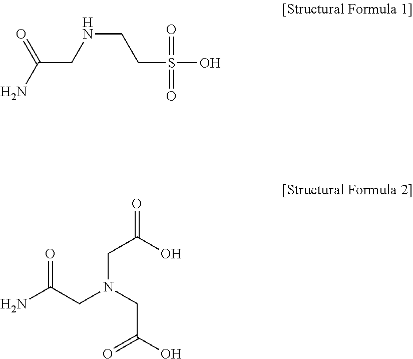 [SMILES: NC(=O)CNCCS(=O)(=O)O ; NC(=O)CN(CC(=O)O)CC(=O)O]

As shown in Structural Formulas 1 and 2, the ACES and ADA buffers include amine groups may bind to the chlorine ions of the sample, thereby obtaining the effect of diluting the sample.

The mechanism by which the ACES and ADA buffers capture the chlorine ions is the same as the mechanism described above with regard to urea.

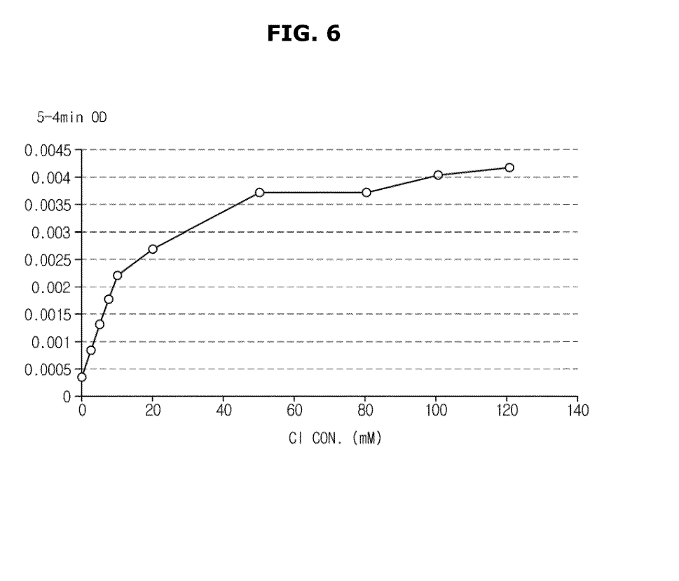

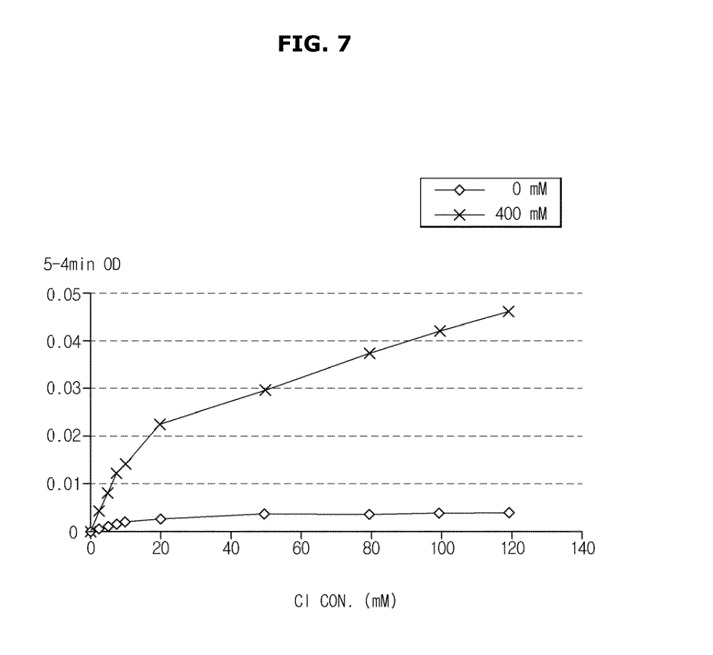

FIG. 6 is an absorbance graph obtained by adding thio-urea according to the method of testing a sample in accordance with an exemplary embodiment, and FIG. 7 is a graph showing a comparison between performing the method of testing including adding thio-urea and not adding thio-urea.

The absorbances shown in FIGS. 6 and 7 are measured by adding a capturing material, α-amylase, and CNPG3 to the sample containing chlorine ions in accordance with the above-described exemplary embodiment. In this instance, 400 mM of thio-urea was used as the capturing material.

Referring to FIG. 6, it can be seen that discrimination between concentrations is enhanced by changing the absorbance shown in a dynamic range by adding 400 mM of the thio-urea to the sample. For example, a concentration ranging from 80 to 135 mM within a range of approximately 0.37 to 0.045.

The graph of FIG. 7 provides a clearer comparison with when the capturing material is not added. As shown in FIG. 7, the absorbance when 400 mM of thio-urea is added has a larger slope in the dynamic range than that when the thio-urea is not added. Thus, according to the method of testing a sample in accordance with an exemplary embodiment, the concentration of chlorine ions within the sample may be more precisely determined without the need for a separate step for diluting the sample.

An exemplary embodiment of a microfluidic device according to one aspect will be described below. The microfluidic device may be used to execute the method of testing a sample.

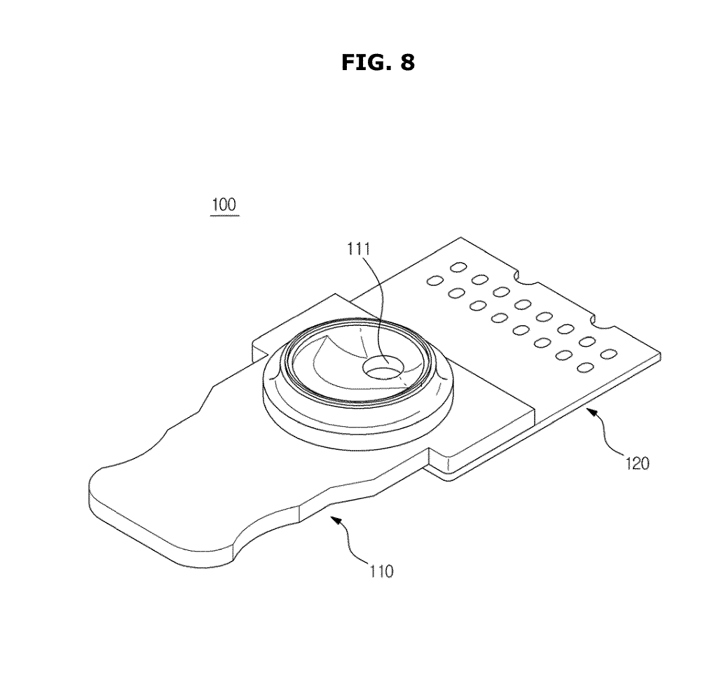

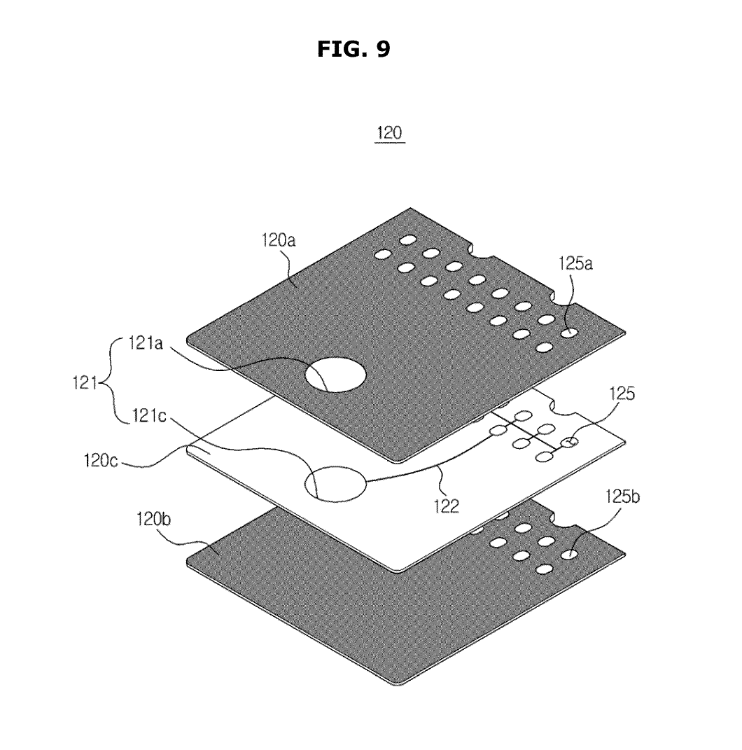

FIG. 8 is an exterior view of a microfluidic device in accordance with an exemplary embodiment, and FIG. 9 is an exploded perspective view of a structure of a testing unit of the microfluidic device shown in FIG. 8.

Referring to FIG. 8, a microfluidic device 100 in accordance with an exemplary embodiment includes a housing 110 and a testing unit 120 within which a sample mixes and reacts with a reagent.

The housing 110 supports the testing unit 120 and allows a user to hold the microfluidic device 100. The housing 110 may be easily molded and formed of a chemically and biologically inactive material.

For example, the housing 110 may be formed from one or more of various materials including an acryl such as polymethylmethacrylate (PMMA), a polysiloxane such as polydimethylsiloxane (PDMS), a polycarbonate (PC), a polyethylene such as a linear low-density polyethylene (LLDPE), a low-density polyethylene (LDPE), a medium-density polyethylene (MDPE), or a high-density polyethylene (HDPE), a polyvinylalcohol, a very low-density polyethylene (VLDPE), a polypropylene (PP), acrylonitrile butadiene styrene (ABS), a plastic material such as a cyclo olefin copolymer (COC), glass, mica, silica, and a semiconductor wafer.

The housing 110 includes a sample provider 111 to receive and supply a fluid sample. Exemplary fluid samples that may be analyzed in the microfluidic device 100, include but are not limited to, a biological sample such as body fluids including blood, tissue fluid, lymph fluid and urine, or an environment sample for water purity control or soil management, and the exemplary target material subjected to detection may be chlorine ions present in the sample.

The testing unit 120 may be connected below the fluid provider 111 of the housing 110, or inserted into a predetermined groove formed in the housing 110 to be connected to and provide fluid communication with the housing 110.

The sample supplied through the sample provider 111 flows into the testing unit 120 through the sample inlet 121 formed in the testing unit 120. Although not shown in FIG. 8, a filter may be disposed between the sample provider 111 and the sample inlet 121 to filter the sample supplied through the sample provider 111. The filter may be a porous polymer membrane formed of a PC, polyethersulfone (PES), polyethylene (PE), polysulfone (PS), or polyacrylsulfone (PASF).

For example, when blood is provided as a sample, blood cells may be filtered from the blood sample through the filter, thereby allowing only blood plasma or serum to flow into the testing unit 120.

Referring to FIG. 9, the testing unit 120 may have a structure in which three plates, 120a, 120b, and 120c are joined. The three plates may be classified as an upper plate 120a, a lower plate 120b and a middle plate 120c. The upper plate 120a and the lower plate 120b may be printed with a light shielding ink to protect the sample flowing therein from external light.

The upper and lower plates 120a and 120b may be formed from a thin film. Exemplary films useful to form the upper and lower plates 120a and 120b include but are not limited to a polyethylene film formed of a VLDPE, LLDPE, LDPE, MDPE, or HDPE, a PP film, a polyvinylchloride (PVC) film, a polyvinyl alcohol (PVA) film, a PS film, and a polyethylene terephthalate (PET) film.

The middle plate 120c of the testing unit 120 may be formed from a porous sheet such as cellulose to serve as a vent. The porous sheet may be formed from a hydrophobic material or subjected to hydrophobic treatment to ensure that the material does not have an influence on the transfer of the sample.

Formed in the testing unit 120 may be the sample inlet 121, a channel 122 through which the sample flows, and one or more reagent chambers 125 within which a reaction between the sample and the reagent occurs. As shown in FIG. 9, when the testing unit 120 is formed in a triple-layer structure, an upper plate hole 121a corresponding to the sample inlet 121 is formed in the upper plate 120a, and one or more portions 125a corresponding to the one or more reagent chambers 125 may be treated to become transparent.

In addition, in the lower plate 120b, one or more portions 125b corresponding to the one or more reagent chambers 125 may be treated to become transparent. The transparency treatment of parts 125a and 125b may be performed so that the optical characteristics resulting from the reaction occurring in the one or more reagent chambers 125 can be measured.

In the middle plate 120c, a middle plate hole 121c corresponding to the sample inlet 121 is formed. Thus, when the upper plate 120a, the middle plate 120c and the lower plate 120b are joined, the upper plate hole 121a overlaps the middle plate hole 121c, thereby forming the sample inlet 121 of the testing unit 120.

The one or more reagent chambers 125 may be formed in the middle plate 120c on an opposite side of the middle plate 120c, as compared to the middle plate hole 121c. The one or more reagent chambers 125 in the middle plate 120c may be formed by removing corresponding portions of the middle plate 120c in a certain shape, such as a circular or square shape. Thus, when the upper plate 120a, the middle plate 120c and the lower plate 120b are joined, the one or more reagent chambers 125 are formed.

The channel 122 may have a width of about 1 to 500 μm, and may be formed in the middle plate 120c to allow the sample to flow to the one or more reagent chambers 125 by capillary action. However, the width of the channel 122 is merely an example applied to the exemplary microfluidic device 100, and the various embodiments described herein are not limited thereto.

A reagent used to detect a target material may be previously loaded into and contained within the one or more reagent chambers 125. Thus, when the target material is chlorine ions, the capturing material may include an amine group that binds to the chlorine ions, and a reagent that changes optical characteristics according to the concentration of the chlorine ions may be contained therein. As a specific example, an enzyme activated by chlorine ions, such as α-amylase, and a substrate degraded by the activated enzyme, such as CNPG3, may be used as the reagents, and urea, thio-urea, an ACE buffer or an ADA buffer may be used as the capturing material.

In various exemplary embodiments, a liquid-phase reagent may be coated on the one or more portions 125a of the upper plate 120a and/or on the one or more portions 125b of the lower plate 120b and dried. Thus, when the upper plate 120a, the lower plate 120b and the middle plate 120c are joined, the reagent is contained within the one or more reagent chambers 125 in a dried state.

In various exemplary embodiments, a single reagent or a combination of two or more kinds of reagents may be used. One kind of reagent may include a capturing material, an enzyme and a substrate may be contained in one of the reagent chambers 125, while a reagent not containing a capturing material may be contained in another of the reagent chambers 125. Thus, an enzyme and a substrate may be included in at least one of the reagents that includes a capturing material, and may also be included in a reagent not including a capturing material. In the exemplary embodiment provided herein, there is no limitation to the type or number of reagents as long as a capturing material, an enzyme and a substrate are contained in the one or more reagent chambers 125.

When the sample including chlorine ions is loaded into the sample provider 111 of the microfluidic device 100, the sample flows into the testing unit 120 through the sample inlet 121 and is thereafter transferred to the one or more reagent chambers 125 through the channel 122.

The sample is then mixed with certain amounts of a capturing material, α-amylase and CNPG3 within the reagent chamber 125, and as shown in FIGS. 4 and 5, after a certain amount of the capturing material binds to a certain amount of chlorine ions present in the sample, the unbound chlorine ions activate the α-amylase. The activated α-amylase then hydrolyzes the CNPG3, thereby generating CNP.

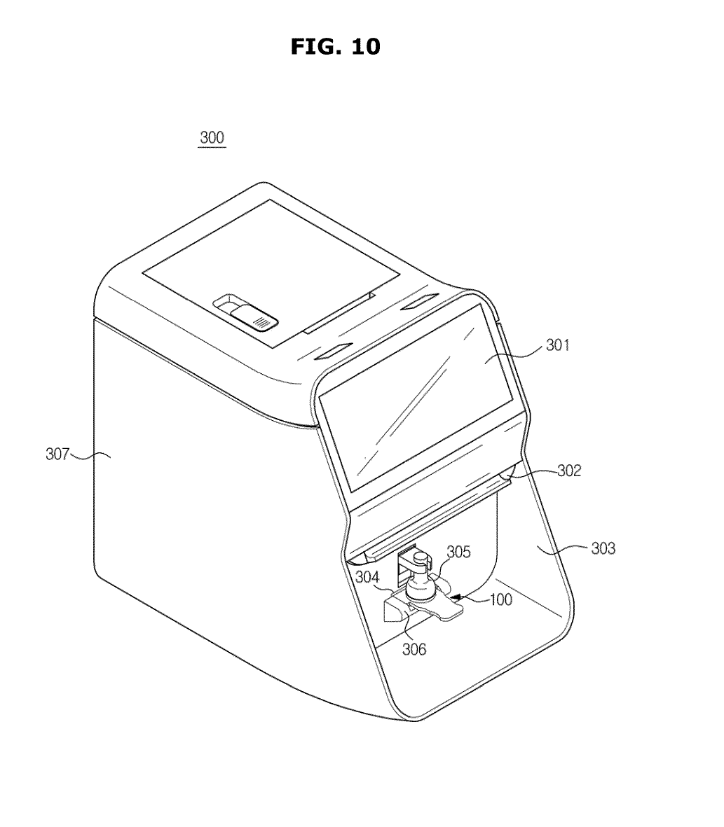

FIG. 10 is an exterior view of a testing device 300 capable of measuring test results using the microfluidic device 100 in accordance with an exemplary embodiment.

The testing device 300 may be a compact and automated device capable of being used to test various types of samples including an environmental sample, a bio sample, a food sample, etc. Particularly, when the device is used in in vitro diagnosis for testing a biological sample, the in vitro diagnosis may be instantly performed by any user, for example, a patient, a doctor, a nurse, or a medical laboratory technologist in any place, for example, at home, a workplace, an outpatient clinic, a patient room, an emergency room, a surgical ward, or an intensive care unit.

Referring to FIG. 10, the testing device 300 includes an installation unit 303, which is a space within which the microfluidic device 100 is installed. When a door 302 of the installation unit 303 slides upward to open, the microfluidic device 100 may be installed in the testing device 300. Specifically, the testing unit 120 of the microfluidic device 100 may be inserted into a predetermined insertion groove 304 formed in the installation unit 303.

The testing unit 120 may therefore be inserted into a main body 307 of the testing device 300, with the housing 110 being exposed to an outside of the testing device 300 and supported by a support 306. In addition, when a pressure unit 305 presses the sample provider 111, the flow of the sample into the testing unit 120 may be stimulated.

After installing the microfluidic device 100 into the installation unit 303, the door 302 is closed, and a test starts. Although not shown in FIG. 10, a detector including a light emission unit and a light reception unit is disposed within the main body 307. The detector radiates light at a specific wavelength to the one or more reagent chambers 125, and detects light transmitted through or reflected from the one or more reagent chambers 125. The wavelength of the radiated light may be determined by the type of material used to produce a change in optical characteristics according to the concentration of the target material.

The testing device 300 may obtain and store optical data resulting from optical characteristics such as absorbance, transmittance, luminance and reflectivity from a signal output from the detector. The optical data may then be used to determine the concentration of chlorine ions present in the sample.

For example, absorbance data may show changes in absorbance over time. In addition, the concentration of a target material may be determined using preloaded information about the absorbance and the concentration of the target material. As an example, the preloaded information on the absorbance and the concentration of the target material may be stored in the form of a calibration curve.

Since the capturing material such as urea, thio-urea, an ACE buffer or an ADA buffer binds to a certain amount of chlorine ions present in the sample, as shown in FIG. 6, discrimination of the concentration may be enhanced even in a concentration range such as the dynamic range.

After the concentration of the chlorine ions is determined by the testing device 300, the results are shown on a display 301.

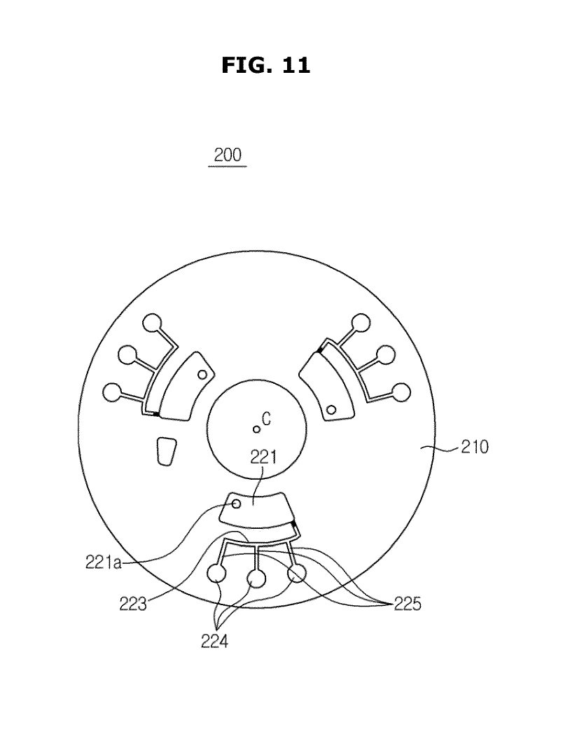

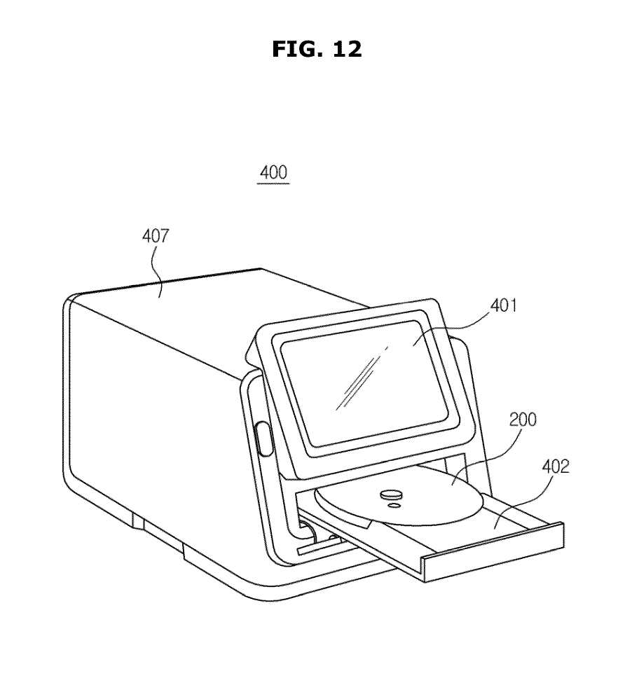

FIG. 11 is a top view of a microfluidic device in accordance with another exemplary embodiment, and FIG. 12 is an exterior view of a testing device for measuring test results using the microfluidic device in accordance with another exemplary embodiment.

Referring to FIG. 11, a microfluidic device 200 in accordance with another exemplary embodiment may be composed of a rotatable platform 210 with microfluidic structures formed therein. The microfluidic structures may include a plurality of chambers 224 containing reagents, and channels 225 connecting these chambers.

The platform 210 may be formed of a material that is easily molded and that has a biologically inactive surface, for example, a plastic material such as PMMA, PDMS, PC, PP, PVA, or PE, glass, mica, silica, or a silicon wafer.

However, in the exemplary embodiment provided herein, any material having chemical and biological stability and mechanical processability may be used to form the platform 210 without limitation, and when test results in the microfluidic device 200 are optically analyzed, the platform 210 may be optically transparent.

The microfluidic device 200 may allow materials in the microfluidic structures to be transferred using centrifugal force. As shown in FIG. 11, a disc-shape platform 210 is exemplified. However, the platform 210 may be formed in an intact disc or fan shape, or could be a polygonal shape as long as it can rotate on a rotatable platform.

In the exemplary embodiment provided herein, the term “microfluidic structures” inclusively refers to chambers and/or channels formed within the platform 210, rather than to a particular structure with a specific shape, and may also include a material serving a specific function as needed. The microfluidic structures may serve different functions depending on dispositional characteristics or the types of materials contained therein.

As shown in FIG. 11, the platform 210 includes a sample inlet 221a, a sample chamber 221 configured to contain the sample and then transfer the sample to another chamber, one or more reagent chambers 224 within which a reaction between a reagent and the sample occurs, and a distribution channel 223 configured to distribute the sample into each of the one or more reagent chambers 224. In addition, although not shown in FIG. 11, when blood is used as the sample, a microfluidic structure for centrifugation of the blood may also be provided within the microfluidic device 200.

As shown in FIG. 11, when a plurality of reagent chambers 224 are included, a plurality of branch channels 225 may branch off from the distribution channel 223 to connect the distribution channel 223 with each of the respective reagent chambers 224.

Reagents including a capturing material binding a target material, an enzyme activated by the target material, and a substrate degraded by the activated enzyme may be contained within each of the one or more reagent chambers 224.

As described in the above exemplary embodiments, when the target material is chlorine ions, a reagent whose optical characteristics change according to a concentration of the chlorine ions may be contained therein. Specifically, an enzyme activated by the chlorine ions, such as α-amylase, and a substrate degraded by the activated enzyme, such as CNPG3, may be used with a capturing material including an amine group, such as urea, thio-urea, an ACE buffer, or an ADA buffer.

The platform 210 may be formed from a plurality of plates. For example, when the platform 210 is formed from two plates, for example, an upper plate and a lower plate, an engraved microfluidic structure, such as a chamber or channel may be formed in a surface on which the upper and lower plates are in contact with each other. Thus when the two plates are joined, a space capable of containing a fluid within the platform 210 and a pathway through which the fluid can be transferred are formed. The joining of the plates may be performed through any of various methods including, but not limited to, adhesion using an adhesive or a double-side tape, ultrasonic fusion, laser welding, etc.

Accordingly, a reagent including a capturing material, an enzyme and a substrate may be contained in various portions of the upper and/or lower plate having the engraved structure corresponding to the reagent chamber 224, and then the upper and lower plates may be joined. As described above, before joining the upper and lower plates, the contained reagent can be dried.

In various embodiments, a single reagent or a combination of two or more types of reagents may be used. One type of reagent may include a capturing material, an enzyme and a substrate may be contained in the reagent chambers 224, or a reagent including a capturing material and a reagent not including a capturing material may be contained in the respective reagent chambers 224. The enzyme and the substrate may be included in at least one of the reagents including a capturing material, and may also be included in a reagent not including a capturing material. In the exemplary embodiment provided herein, there is no limitation on the type or number of the reagents as long as a capturing material, an enzyme and a substrate are contained in the reagent chamber 224.

In FIG. 11, the reaction between the sample and the reagent occurs in one or more of the reaction chambers 224 containing the reagent. However, in various embodiments, the microfluidic device 200 may have a separate chamber in which the reaction occurs when the reagent and the sample are transferred. In addition, a capturing material, an enzyme and a substrate may not be contained in a single reagent chamber 224. In certain embodiments at least one of them is contained in the reagent chamber 224, and then transferred to the chamber in which the reaction between the sample and the reagent occurs during a test.

In a specific process of the test, the sample including chlorine ions is injected into the sample providing chamber 221 through the sample inlet 221a of the microfluidic device 200, and as shown in FIG. 12, the microfluidic device 200 is placed on a tray 402 of the testing device 400. The microfluidic device 200 is then inserted into the main body 407 of the testing device 400 when the tray 402 retracts therein. The testing device 400 may then rotate the microfluidic device 200 according to a sequence determined by the type of microfluidic device and/or the type of test to be performed. The sample injected into the sample providing chamber 221 may then be transferred in a direction away from the center (C) of rotation by centrifugal force.

A valve may be disposed at any one or more of an opening of the reagent chamber 224, an outlet of the sample providing chamber 221, a point of the distribution channel 223, or a point of the branch channel 225. When the valve is open, the sample flows into the reagent chamber 224 and reacts with certain amounts of a capturing material, α-amylase and CNPG3. As discussed above, a certain amount of the chlorine ions present in the sample bind to the capturing material, and the rest of the chlorine ions activate the α-amylase. The activated α-amylase hydrolyzes the CHPG3, thereby generating CNP.

As discussed above, a detector including a light emission unit and a light reception unit is included within the main body 407, and is configured to radiate light to the reagent chamber 224 of the microfluidic device 200, and to detect light transmitting or reflected from the one or more reagent chambers 125.

Optical data resulting from optical characteristics such as absorbance, transmittance, luminance and reflectivity from a signal output from the detector may be obtained and stored in the test device 400. The optical data may then be sued to determine a concentration of chlorine ions present in the sample as described above. Since the capturing material binds to a certain amount of chlorine ions in the sample, as shown in FIG. 6, enhanced concentration discrimination may be ensured even in a dynamic range of a concentration range.

According to the above-described exemplary embodiments, concentration discrimination in a dynamic range may therefore be enhanced without the need for a separate step or structure for diluting the sample.

Although a few exemplary embodiments have been shown and described, it would be appreciated by those skilled in the art that changes may be made in these embodiments without departing from the principles and spirit of the inventive concept, the scope of which is defined in the claims and their equivalents.

What is claimed is:

1. A method of determining the concentration of chlorine ions in a sample, the method comprising:
mixing a sample with a reagent that changes optical characteristics in accordance with a concentration of chlorine ions in the sample, and a capturing material that captures some of the chlorine ions in the sample;
measuring the optical characteristics after mixing the sample with the reagent and the capturing material; and
determining a concentration of the chlorine ions in the sample based on the measured optical characteristics.

2. The method according to claim 1, wherein the capturing material is a compound comprising an amine (—NH₂) group.

3. The method according to claim 2, wherein the capturing material comprises at least one selected from the group consisting of urea, thio-urea, an N-(2-acetamido)-2-aminoethanesulfonic acid (ACES) buffer, and a 2-[(2-amino-2-oxoethyl)-(carboxymethyl)amino]acetic acid (ADA) buffer.

4. The method according to claim 2, wherein the amine group of the capturing material binds to the chlorine ions.

5. The method according to claim 2, wherein the reagent comprises an enzyme activated by the chlorine ions and a substrate degraded by the activated enzyme.

6. The method according to claim 5, wherein the enzyme is activated by chlorine ions that are not bound to the capturing material.

7. The method according to claim 6, wherein the enzyme is α-amylase.

8. The method according to claim 7, wherein the substrate is 2-chloro-4-nitrophenyl-alpha-maltotrioside (CNPG3).

9. The method according to claim 8, wherein the CNPG3 is hydrolyzed by the α-amylase to generate 2-chloro-4-nitrophenol (CNP) and α-maltotriose (G3).

10. A microfluidic device comprising:
at least one chamber containing a reagent that changes optical characteristics according to a concentration of chlorine ions in a sample, and a capturing material that captures some of the chlorine ions in the sample; and
a sample inlet into which the sample is injected.

11. The device according to claim 10, wherein the capturing material is a compound comprising an amine (—NH₂) group.

12. The device according to claim 11, wherein the capturing material comprises at least one selected from the group consisting of urea, thio-urea, an N-(2-acetamido)-2-aminoethanesulfonic acid (ACES) buffer, and a 2-[(2-amino-2-oxoethyl)-(carboxymethyl)amino]acetic acid (ADA) buffer.

13. The device according to claim 11, wherein the amine group of the capturing material binds to the chlorine ions.

14. The device according to claim 11, wherein the reagent comprises an enzyme activated by the chlorine ions and a substrate degraded by the activated enzyme.

15. The device according to claim 14, wherein the enzyme is activated by chlorine ions that are not bound to the capturing material.

16. The device according to claim 15, wherein the enzyme, substrate and capturing material are contained in one of the at least one chambers.

17. The device according to claim 16, further comprising:
a channel connecting the chamber containing the enzyme, substrate and capturing material with the sample inlet.

18. The device according to claim 15, wherein the enzyme is α-amylase.

19. The device according to claim 18, wherein the substrate is 2-chloro-4-nitrophenyl-alpha-maltotrioside (CNPG3).
